(19) United States
(12) Patent Application Publication
(10) Pub. No.: US 2022/0155206 A1
(43) Pub. Date: May 19, 2022

(54) METHOD FOR ESTABLISHING A CHARACTERISTIC ATLAS OF WHOLE IMMUNE CELLS IN LUNGS OF MICE WITH ACUTE LUNG INJURY

(71) Applicant: Zhejiang University, Hangzhou (CN)

(72) Inventors: Hongcui Cao, Hangzhou (CN); Lanjuan Li, Hangzhou (CN); Jiaqi Zhu, Hangzhou (CN); Jiong Yu, Hangzhou (CN)

(21) Appl. No.: 17/561,761

(22) Filed: Dec. 24, 2021

Related U.S. Application Data
(63) Continuation-in-part of application No. PCT/CN2020/094897, filed on Jun. 8, 2020.

(30) Foreign Application Priority Data
Jun. 24, 2019 (CN) 201910551631.5

(51) Int. Cl. G01N 15/10; G01N 1/30; G01N 1/40

(52) U.S. Cl. CPC G01N 15/1031; G01N 1/30; G01N 2001/302; G01N 2015/1006; G01N 1/4077

(57) ABSTRACT

The present invention provides a method for establishing a characteristic atlas of whole immune cells in lungs of mice with acute lung injury, including: extraction of the whole immune cells in lung; labeling of antibody with specific metal isotope for mass cytometry; staining the immune cells with the detection antibody; and mass cytometer analysis. The present invention is capable of isolating whole immune cells with high yield and viability on the basis of ensuring cell purity, greater than that of the conventional grinding method. The present invention binds stable metal isotopes to antibodies, while mass spectrometry flow channels are designed based on the principle of minimal channel interference to achieve a comprehensive description on the classification and function of whole immune cells in lung of the mouse with up to 43 markers simultaneously; dynamic alterations in the whole immune cells in lung of the mouse can also be observed.

### CROSS-REFERENCE TO RELATED APPLICATION

The present application is a Continuation-in-part Application of PCT Application No. PCT/CN2020/094897 filed on Jun. 8, 2020, which claims the benefit of Chinese Patent Application No. 201910551631.5 filed on Jun. 24, 2019. The contents of the above are hereby incorporated by reference in their entirety.

### TECHNICAL FIELD

The present invention relates to a study of a characteristic atlas of whole immune cells in lungs, and provides a method for mapping a characteristic atlas of whole immune cells in lungs of mice with acute lung injury.

### BACKGROUD

Acute lung injury is a life-threatening lung disease in which massive epithelial and/or endothelial cell damage occurs in a short period of time, inducing an inflammatory response. Endothelial dysfunction and local inflammation lead to diffuse alveolar injury, resulting in severe hypoxemia and inflammatory infiltration in lungs. Severe lung injury can rapidly progress to respiratory distress or respiratory failure within hours or days, with high morbidity and mortality. Although there have been many basic and clinical studies on acute lung injury, the molecular regulatory mechanism of immune response to acute lung injury remain unclear. In recent years, the therapeutic means targeting pathogenesis thereof, for example, immune agents, stem cell transplantation and so on, have developed rapidly and become a potential new therapeutic tool.

Immune cells, such as B lymphocytes, T lymphocytes and so on, in acute lung injury are heavily infiltrated in lung, and at the same time, the immune cells secrete a large amount of pro-inflammatory factors, which further aggravates the lung injury. In a pathological state of lung injury, the whole immune cells in lung have changes in terms of the type and proportion, and these changes are important for the development of novel immunomodulatory drugs. Due to a limitation of current techniques and methods, the characteristic changes of lung intrinsic and reactive immune cells during lung injury remain unclear. In conventional isolation method, the immune cells in lung are labeled with an antibody for flow cytometry and isolated by conventional flow cytometry. However, it is difficult to perform experiments with more than 12 colors because of the technical limitation of the conventional flow cytometry method such as limited detection channels and overlap of emission spectra of fluorophores, resulting in insufficient coverage of the detection of immune cell in lung, which cannot accurately reflect the characteristic changes of immune cell subpopulations in lung during lung injury.

### SUMMARY

A technical problem to be solved by the present invention is to overcome the deficiency in the prior art and provide a method for establishing a characteristic atlas of whole immune cells in lungs of mice with acute lung injury.

The present invention provides a technical solution to solve the technical problem.

Provided is a method for establishing a characteristic atlas of whole immune cells in lungs of mice with acute lung injury, comprising:

(1) Extraction of the whole immune cells in lung from the mouse

Taking a fresh mouse carcass that died naturally due to acute lung injury and removing the lung after irrigation and drainage of blood inside the lung; cutting up and digesting the lung with an enzyme mixture for half an hour, then performing density gradient centrifugation and erythrocyte lysis to obtain pure whole immune cells in lung of the mouse;

(2) Labeling of antibody for mass cytometry

Binding the antibody against an immune cell marker in lung of the mouse with a stable metal isotope by using the MaxPAR X8 antibody coupling kit from Fluidigm, USA, to obtain a labeled antibody;

(3) Staining the immune cells with the antibody

Incubating the isolated whole immune cells in lung of the mouse with the labeled antibody to label the immune cells;

(4) Mass cytometry analysis

Loading and analyzing the labeled whole immune cells in lung of the mouse on a mass cytometry, and analyzing the obtained data by t-SNE and X-shift algorithms; then distributing expressions of a plurality of detection antibodies of different cell subpopulations on a same heat map, and representing expressions of different markers and a distribution of different cell subpopulations by viSNE map, thereby showing a classification atlas of the whole immune cells in lung of the mouse.

In the present invention, the step (1) specifically comprises:

(1.1) Wiping the mouse carcass with a 75% ethanol cotton ball and cutting open the chest;

(1.2) Continuously perfusing and flushing the lung with phosphate buffer saline (PBS) through the heart, and removing blood inside the lung to change the lung from blood red to white;

(1.3) Isolating the lung from the mouse and placing in a culture dish containing PBS for washing by immersion;

(1.4) Cutting up the lung and placing in a dissociation tube containing 2.4 mL of dulbecco's modified eagle medium (DMEM) and 0.3 mL of enzyme mixture;

(1.5) Placing the dissociation tube into a GentleMACS™ Dissociator dissociation machine from MACS, Germany, and running two times in a m_lung_01 mode;

(1.6) Removing the dissociation tube and placing on a shaker at a constant temperature of 37° C. and 220 rpm for 30 minutes for digestion;

(1.7) After digestion, placing the dissociation tube in the dissociation machine again and running one time in a m_lung_02 mode to obtain a suspension;

(1.8) Filtering the suspension in the dissociation tube through a 100 μm filter into a 15 mL centrifuge tube, resuspending the dissociation tube with 2.5 mL of PBS to obtain a liquid portion, and filtering the liquid portion into the centrifuge tube again;

(1.9) Centrifuging at a relative centrifugal force of 300 g for 10 minutes at room temperature to obtain a first supernatant and a first precipitate;

(1.10) Discarding the first supernatant to obtain the first precipitate, and adding 3 mL of 36% Percoll cell separation solution to the first precipitate; centrifuging at a relative centrifugal force of 450 g for 5 minutes at room temperature to obtain a second supernatant containing lung cell debris and a second precipitate;

(1.11) Discarding the second supernatant containing lung cell debris to obtain the second precipitate and adding 3 mL of ACK lysis buffer to the second precipitate for 3 minutes to completely remove erythrocytes; then adding 5 mL of PBS to terminate lysis of erythrocytes and centrifuging at a relative centrifugal force of 400 g for 5 minutes at 4° C. to obtain a third supernatant and whole immune cells; discarding the third supernatant to obtain the pure whole immune cells in lung of the mouse.

In the present invention, the PBS contains sodium chloride, potassium dihydrogen phosphate, disodium hydrogen phosphate and potassium chloride at a total concentration of 0.01 mol/L and a pH value of 7.4.

In the present invention, the enzyme mixture is prepared by adding 12 mg of collagenase IV, 30 mg of pronase and 5 mg of deoxyribonuclease I powder into 100 mL of PBS and mixing well.

In the present invention, the dissociation tube containing the dulbecco's modified eagle medium and the enzyme mixture is preheated in a water bath at 37° C. for 5 minutes prior to placing the cut lung in the dissociation tube.

In the present invention, the Percoll cell separation solution is prepared by mixing 1 mL of 10× PBS with 9 mL of original Percoll solution well and then adding 15 mL of 1× PBS to obtain 25 mL of 36% Percoll separation solution.

In the present invention, the 10× PBS contains sodium chloride, potassium dihydrogen phosphate, disodium hydrogen phosphate and potassium chloride at a total concentration of 0.1 mol/L and a pH value of 7.4.

In the present invention, the centrifuge should be adjusted both ascending and descending speeds to the lowest setting before turning on the centrifuge in the steps (1.9) and (1.10).

In the present invention, the step (2) specifically comprises: labeling a multimer with a metal marker to obtain a multimer chelated to a specific metal firstly, and then labeling an antibody with the multimer chelated to the specific metal to obtain the labeled antibody.

In the step (2) of the present invention, the stable metal isotope comprises: yttrium (Y-89), indium (In-113, In-115), lanthanum (La-139), praseodymium (Pr-141), neodymium (Nd-142, Nd-143, Nd-144, Nd-145, Nd-146, Nd-148, Nd-150), samarium (Sm 147, Sm-149, Sm-152, Sm-154), europium (Eu-151, Eu-153), gadolinium (Gd-155, Gd-156, Gd-157, Gd-158, Gd-160, Gd-197), terbium (Tb-159), dysprosium (Dy-161, Dy-162, Dy-163, Dy-164), holmium (Ho-165), Erbium (Er-166, Er-167, Er-168, Er-170), Thulium (Tm-169), Ytterbium (Yb-171, Yb-172, Yb-173, Yb-174, Yb-176), Lutetium (Lu-175), Platinum (Pt-198), Bismuth (Bi-209);

the antibody comprises 43 antibodies, comprising: anti-CD45, anti-CD44, anti-CD19, anti-KI67, anti-CD24, anti-MHC II, anti-B220, anti-CDS, anti-CD43, anti-CD38, anti-Ly6G, anti-Ly6C, anti-CX3CR1, anti-IgD, anti-CD62L, anti-CD11c, anti-TCRγδ, anti-CD49a, anti-CD80, anti-BST2, anti-CD25, anti-CD3, anti-F4/80, anti-CD115, anti-iNOS, anti-CXCR3, anti-CD27, anti-CD103, anti-ICOS, anti-Argnase I, anti-CD49b, anti-Foxp3, anti-CD127, anti-CD21, anti-CD23, anti-CD138, anti-CD172a, anti-CTLA-4, anti-SiglecF, anti-IgM, anti-CD4, anti-CD8a, anti-CD11b.

The beneficial effects of the present invention compared with the prior art are showed as below.

1. In the conventional separation method, a lung tissue is ground and centrifuged depending to a cell density, resulting in that such separation gives a low yield of immune cells, and only a specific density of cell subpopulations, not all immune cells in lungs, can be effectively isolated. However, the method of the present invention can isolate the whole immune cells in lungs of mice with high yield on the basis of ensuring cell purity.

2. The flow cytometry is used to verify whether the cells are bound to propidium iodide and CD45-fluorescein isothiocyanate, wherein the cell is a dead cell when showing propidium iodide positive and is an immune cell when showing CD45 positive. Compared with the results obtained by grind and isolation in the prior art, the yield of the whole immune cells in lungs of mice isolated by the present invention is greater than 5×10⁶ per mouse, which was higher than the yeild of 1-2.5×10⁶ per mouse by the grinding method, and the cell viability is greater than 95%, which is greater than the cell viability of about 85% by the conventional grinding method.

3. The MaxPAR X8 antibody coupling kit is a commercial product, which contains only a metal isotope and a coupling reagent. In the present invention, the kit is innovatively applied to bind the stable metal isotope with a purified commercial antibody product, based on the characteristics of immune cells in the lung, while the classification and function of the whole immune cells in lungs of mice are comprehensively described by designing a mass spectrometry flow cytometry channel based on the principle of minimal channel interference.

4. The experiment greater than 12 colors is difficult to be performed by the conventional flow cytometry assay technique, resulting in insufficient coverage of the detection of immune cells in lung which cannot accurately reflect the characteristic changes of immune cell subpopulations in lung during acute lung injury. The method in the present invention is able to systematically detect and classify the immune cells in lung of mouse with up to 43 markers simultaneously, including lineage specific surface markers, cytokine markers and functional markers, such as co-stimulatory molecular markers.

5. The dynamic changes of the whole immune cells in lungs of mice can be observed in the present invention.

### BRIEF DESCRIPTION OF THE DRAWINGS

FIG. 1 is a microscopic image of the morphology of the isolated immune cells in lung (observed by 20× object lens).

FIG. 2 is a wright staining image of the isolated immune cells in lung.

FIG. 3 is a classification atlas of the whole immune cells in lung of mouse with acute lung injury.

FIG. 4 is an expression of markers of each group of immune cells in lungs of mice with acute lung injury (heatmap).

### DETAILED DESCRIPTION

The source of the mouse lungs used in the present invention is first described.

The mouse lungs used in the present invention were taken from laboratory discarded mice that died naturally due to acute lung injury, and the lungs should be removed within 0.5 hours from the time of death. C57BL/6J pup mouse carcasses, with 6-8-week age and a weight of 18-25 g, were selected, and washed with 100 mL of 75% ethanol. The lungs were removed in a biosafety cabinet for subsequent testing. In the implementation of the present invention, there is no operation to kill a surviving mouse and no traumatic or interventional disposal such as dissection or excision on a live mouse.

The technical solutions of the present invention are described in detail below in combination with specific implementation examples.

Apparatus and reagents

Dissociation tube (MACS, Germany), dissociation machine (MACS, Germany), thermostatic shaker (Thermo, America), centrifuge tube (Corning, America), centrifuge (Eppendorf, Germany), 10 cm culture dish (Greiner, Germany), 100 μm filter (Corning, America), water bath kettle (Thermo, America), inverted microscope (Nikon, Japan), digital slide scanner (HAMAMATSU, Japan), slides (Shitai, China), Mass Cytometer (Fluidigm, America), water purifier (Thermo, America), 50 kilodalton (kDa) filters (merck millipore, America), 3 kilodalton (kDa) filters (merck millipore, America),

DMEM (Gibco, America), PBS (Gino, China), 10× PBS (Gibco, America), collagenase IV (Invitrogen, America), pronase (Roche, America), deoxyribonuclease I (Sigma, America), percoll cell separation solution (GE Healthcare, Sweden), ACK Lysis Buffer (Gibco, America), wright-giemsastain (Beso, China), multimeric linker (Fluidigm, America), metal isotope (Fluidigm, America), MaxPAR X8 antibody coupling kit (Fluidigm, America), bovine serum albumin (Solarbio, China), sodium azide (Sigma, America), TCEP reductant (Thermo, America), antibody blocking solution (Equitech-Bio, America), fixative (Fluidigm, America), perm buffer (Fluidigm, America), 20% EQ beads (Fluidigm, America).

I. Isolation of immune cells in lungs of mice

The whole immune cells in lungs of mice are efficiently obtained by a method comprising the following steps.

(1) Fresh carcasses of mice with acute lung injury that died naturally were taken and wiped with a 75% ethanol cotton ball, and then the thorax thereof was cut open.

(2) The lungs were continuously perfused and flushed with phosphate buffer saline (PBS) through the heart to remove internal blood to change the lungs from blood red to white.

(3) The mouse lungs were isolated and placed in a 10 cm culture dish containing PBS for washing by immersion.

(4) The lungs were cut up and placed in a dissociation tube containing 2.4 mL of dulbecco's modified eagle medium (DMEM) and 0.3 mL of enzyme mixture.

(5) The dissociation tube was placed in a GentleMACS™ Dissociator dissociation machine from MACS, Germany, running two times in a m_lung_01 mode.

(6) The dissociation tube was then placed on a thermostatic shaker rotating at 220 rpm at 37° C. for 30 minutes for digestion.

(7) After digestion, the dissociation tube was placed in the dissociation machine again, running one time in a m_lung_02 mode to obtain a suspension.

(8) The suspension in the dissociation tube was filtered through a 100 μm filter into a 15 mL centrifuge tube, and then the dissociation tube was resuspended with 2.5 mL of PBS before the liquid portion was filtered into a 15 mL centrifuge tube.

(9) The centrifuge tubes were centrifuged for 10 minutes at room temperature at a relative centrifugal force of 300 g to obtain a first supernatant and a first precipitate, taking care to adjust both the ascending and descending speeds of the centrifuge to the lowest setting before centrifugation.

(10) The first supernatant was discarded to obtain the first precipitate, and 3 mL of 36% Percoll cell separation solution was added into the first precipitate and centrifuged at a relative centrifugal force of 450 g for 5 minutes at room temperature to obtain a secondsupernatant containing lung cell debris and a second precipitate, taking care to adjust both the ascending and descending speeds of the centrifuge to the lowest setting before centrifugation.

(11) The second supernatant was discarded to obtain the second precipitate, and 3 mL of ACK lysis buffer was added to the second precipitate for 3 minutes to completely remove the erythrocytes, and then 5 mL of PBS was added thereinto to terminate lysis of erythrocytes and centrifuged at a relative centrifugal force of 400 g for 5 minutes at 4° C. to obtain a third supernatant and whole immune cells. The third supernatant was discarded to obtain pure whole immune cells in lungs of mice.

In the above steps, the PBS is a commercial reagent containing sodium chloride, potassium dihydrogen phosphate, disodium hydrogen phosphate and potassium chloride with a concentration of 0.01 mol/L (potassium dihydrogen phosphate 0.24 g/L, disodium hydrogen phosphate 1.42 g/L, sodium chloride 8.0 g/L, potassium chloride 0.2 g/L) and a pH of 7.4. The enzyme mixture is prepared by adding 12 mg of collagenase IV, 30 mg of pronase and 5 mg of deoxyribonuclease I powder into 100 mL of PBS and mixing well. There was a need to put the dissociation tube containing the dulbecco's modified eagle medium and the enzyme mixture into a water bath at 37° C. for 5 minutes to be preheated. The filter is a 100 μm filter. The 36% Percoll cell separation solution is prepared by mixing 1 mL of 10x PBS with 9 mL of original Percoll solution well and then adding 15 mL of 1× PBS to obtain 25 mL of 36% Percoll separation solution. The 10× PBS had a component consisting of sodium chloride, potassium dihydrogen phosphate, disodium hydrogen phosphate and potassium chloride at a concentration of 0.1 mol/L and a pH of 7.4.

Cell morphology observation

The whole immune cells in lungs of mice obtained in Example I were resuspended with 1 mL of PBS and dropped a drop on the center of the slide, and the cells were observed under an inverted microscope.

Identification of immune cells by wright staining

(1) The immune cells were resuspended with 1 mL of PBS and dropped a drop on the quarter of the whole slide, and the drop was pushed by another slide toward the other end of the slide at a uniform speed, and formed a cell smear after drying.

(2) 0.5 mL of wright-giemsastain A solution was added dropwise onto the smear and the staining solution was allowed to cover the entire specimen for 1 minute.

(3) The wright-giemsastain B solution was then added on top of the A solution (2-3 times the amount of the A solution), and a breeze was blown out by mouth or a washing ear ball to create ripples in the liquid surface, so that the two solutions are fully mixed and stained for 5 minutes.

(4) The slide was washed with water (when washing, the staining solution should not be poured off first, but should be washed off with running water to prevent any sediment from precipitating on the specimen), dried, and then scanned by a digital slide scanner. The nucleus of immune cells can be stained fuchsia and the cytoplasm can be stained pink.

Experimental results

Observation on the morphology of the isolated immune cells

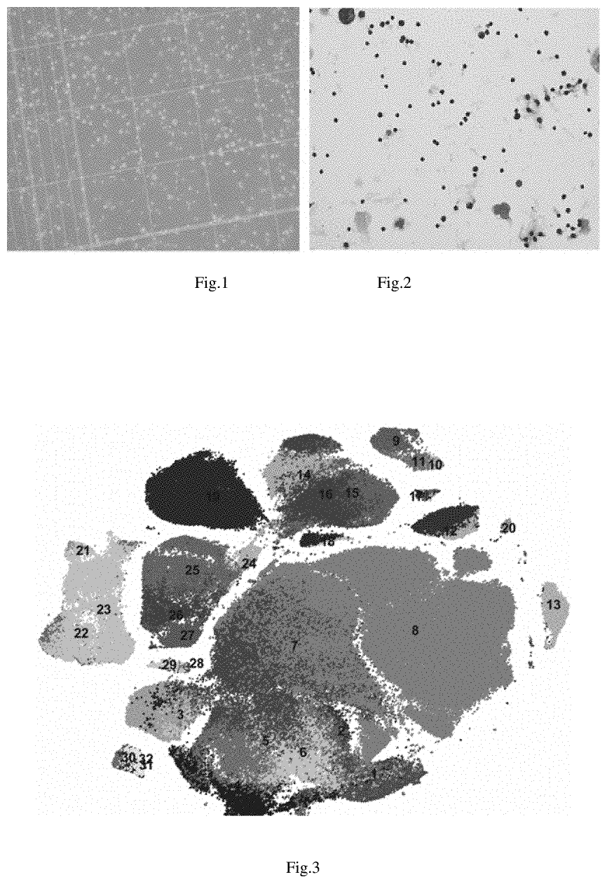

It was found from the observation of the immune cells under an inverted microscope that the freshly isolated cells were round and full with good transparency (observed by 20× object lens), and contained fewer impurities (see FIG. 1).

Identification of the isolated immune cells by wright staining

It was observed that the nucleus of the isolated immune cells was all stained fuchsia, the cytoplasm was stained pink, and most of the cells were mononuclear cell and no erythrocyte was seen, based on the image scanned by the scanner (see FIG. 2).

II. Labeling of antibody for mass spectrometry flow cytometry

(1) 5μl of metal isotopes at a final concentration of 2.5 mmol/L and multimeric linkers were taken, incubated for 30 min at 37° C.

(2) 300 μl of R-buffer was added into a 50 kilodalton (kDa) rotating filter, followed by 100 μg of antibody to the above filter, and centrifuged for 10 minutes at a relative centrifugal force of 12,000 g.

(3) 100 μl of TCEP reducing agent was added, preheated at 37° C. for 30 minutes to incubate and reduce the antibody.

(4) 200 μl of L-Buffer was added to a 3 kilodalton (kDa) rotating filter, and the multimer chelated to a specific metal was transferred to the 3 kilodalton (kDa) rotating filter, and then centrifuged at a relative centrifugal force of 12,000 g for 25 minutes; 300 μl of C-Buffer was further added and centrifuged at a relative centrifugal force of 12,000 g for 30 minutes.

(5) 300 μl of C-Buffer was further added to the 50 kilodalton (kDa) rotating filter and centrifuged at a relative centrifugal force of 12,000 g for 10 minutes; 400 μl of C-Buffer was further added and centrifuged at a relative centrifugal force of 12,000 g for 10 minutes.

(6) The precipitates in the 50 kilodalton (kDa) rotating filter and 3 kDa rotating filter were mixed evenly, then transferred to a 50 kDa rotating filter, and mix well, and then preheated at 37° C. for 90 minutes to incubate and reduce the antibody.

(7) 300 μl of W-Buffer was added into the 50 kilodalton (kDa) filter and centrifuged for 10 minutes at a relative centrifugal force of 12,000 g, repeated for three times.

(8) The labeled antibody was recycled with W-Buffer and its optical density value was measured at 280 nm to calculate the concentration.

The metal isotope, R-buffer, L-Buffer, C-Buffer, and W-Buffer reagents involved in the above steps were obtained from the MaxPAR X8 Antibody Coupling Kit (Fluidigm, America)

The metal isotopes involved in the above steps comprise yttrium (Y-8i9), indium (In-113, In-115), lanthanum (La-139), praseodymium (Pr-141), neodymium (Nd-142, Nd-143, Nd-144, Nd-145, Nd-146, Nd-148, Nd-150), samarium (Sm-147, Sm -149, Sm-152, Sm-154), europium (Eu-151, Eu-153), gadolinium (Gd-155, Gd-156, Gd-157, Gd-158, Gd-160, Gd-197), terbium (Tb-159), dysprosium (Dy-161, Dy-162, Dy-163, Dy-164), holmium (Ho-165), Erbium (Er-166, Er-167, Er-168, Er-170), thulium (Tm-169), ytterbium (Yb-171, Yb-172, Yb-173, Yb-174, Yb-176), lutetium (Lu-175), platinum (Pt-198), bismuth (Bi-209).

The antibodies involved in the above step comprise anti-CD45, anti-CD44, anti-CD19, anti-KI67, anti-CD24, anti-MHC II, anti-B220, anti-CDS, anti-CD43, anti-CD38, anti-Ly6G, anti-Ly6C, anti-CX3CR1, anti-IgD, anti-CD62L, anti-CD11c, anti-TCRγδ, anti-CD49a, anti-CD80, anti-BST2, anti-CD25, anti-CD3, anti-F4/80, anti-CD115, anti-iNOS, anti-CXCR3, anti-CD27, anti-CD103, anti-ICOS, anti-Argnase I, anti-CD49b, anti-Foxp3, anti-CD127, anti-CD21, anti-CD23, anti-CD138, anti-CD172a, anti-CTLA-4, anti-SiglecF, anti-IgM, anti-CD4, anti-CD8a, anti-CD11b.

The coupling of the isotopes with the antibodies in the above steps is shown as follows:

| Metallic isotope | Antibody |
|---|---|
| Y-89 | anti-CD45 |
| In-113 | anti-CD44 |
| In-115 | anti-CD19 |
| La-139 | anti-KI67 |
| Pr-141 | anti-CD24 |
| Nd-142 | anti-MHC II |
| Nd-143 | anti-B220 |
| Nd-144 | anti-CD5 |
| Nd-145 | anti-CD43 |
| Nd-146 | anti-CD38 |
| Sm-147 | anti-Ly6G |
| Nd-148 | anti-Ly6C |
| Sm-149 | anti-CX3CR1 |
| Nd-150 | anti-IgD |
| Eu-151 | anti-CD62L |
| Sm-152 | anti-CD11c |
| Eu-153 | anti-TCRγδ |
| Sm-154 | anti-CD49a |
| Gd-155 | anti-CD80 |
| Gd-156 | anti-BST2 |
| Gd-157 | anti-CD25 |
| Gd-158 | anti-CD3 |
| Tb-159 | anti-F4/80 |
| Gd-160 | anti-CD115 |
| Dy-161 | anti-iNOS |
| Dy-162 | anti-CXCR3 |
| Dy-163 | anti-CD27 |
| Dy-164 | anti-CD103 |
| Ho-165 | anti-ICOS |
| Er-166 | anti-Argnase I |
| Er-167 | anti-CD49b |
| Er-168 | anti-Foxp3 |
| Tm-169 | anti-CD127 |
| Er-170 | anti-CD21 |
| Yb-171 | anti-CD23 |
| Yb-172 | anti-CD138 |
| Yb-173 | anti-CD172a |
| Yb-174 | anti-CTLA-4 |
| Lu-175 | anti-SiglecF |
| Yb-176 | anti-IgM |
| Gd-197 | anti-CD4 |
| Pt-198 | anti-CD8a |
| Bi-209 | anti-CD11b |

The sources of the antibodies in the above steps is shown as follows:

| Antibody | Source |
|---|---|
| anti-CD45 | Fluidigm, America |
| anti-CD44 | Biolegend, America |
| anti-CD19 | Biolegend, America |
| anti-KI67 | eBioscience, America |
| anti-CD24 | Biolegend, America |
| anti-MHC II | BioXcell, America |
| anti-B220 | Biolegend, America |
| anti-CD5 | Biolegend, America |
| anti-CD43 | Biolegend, America |
| anti-CD38 | Biolegend, America |
| anti-Ly6G | Biolegend, America |
| anti-Ly6C | Biolegend, America |
| anti-CX3CR1 | Biolegend, America |
| anti-IgD | Biolegend, America |
| anti-CD62L | Biolegend, America |
| anti-CD11c | Biolegend, America |
| anti-TCRγδ | Biolegend, America |
| anti-CD49a | Biolegend, America |
| anti-CD80 | Biolegend, America |
| anti-BST2 | R&D systems, America |
| anti-CD25 | Biolegend, America |
| anti-CD3 | Biolegend, America |
| anti-F4/80 | Bio-rad, America |
| anti-CD115 | Biolegend, America |
| anti-iNOS | Fluidigm, America |
| anti-CXCR3 | Biolegend, America |
| anti-CD27 | Biolegend, America |
| anti-CD103 | Biolegend, America |
| anti-ICOS | Biolegend, America |
| anti-Argnase I | Fluidigm, America |
| anti-CD49b | Biolegend, America |
| anti-Foxp3 | eBioscience, America |
| anti-CD127 | Biolegend, America |
| anti-CD21 | BD Biosciences, America |
| anti-CD23 | Biolegend, America |
| anti-CD138 | Biolegend, America |
| anti-CD172a | Biolegend, America |
| anti-CTLA-4 | Biolegend, America |
| anti-SiglecF | BD Biosciences, America |
| anti-IgM | Biolegend, America |
| anti-CD4 | Biolegend, America |
| anti-CD8a | Biolegend, America |
| anti-CD11b | Biolegend, America |

III Immune cell labeling

(1) 5×10⁶ of whole immune cells in lung extracted in step (I) were taken, added 1 mL of buffer for flow cytometry detection (FACS Buffer), and centrifuged at a relative centrifugal force of 400 g for 5 min, and then the supernatant was discarded.

(2) 100 μl of PBS containing the metal isotope platinum (Pt-194) (1:4000, i.e., 0.25 μM) was added to resuspend the cells and placed on ice for 5 minutes.

(3) 1 mL of FACS Buffer was added to terminate the reaction and centrifuged at a relative centrifugal force of 400 g for 5 min, and then the supernatant was discarded. Antibody blocking solution was added at a volume ratio of 1:100 and placed on ice for 20 min for blocking.

(4) 50 μl of a mixture of antibodies coupled with the metal isotopes was added and incubated on ice for 30 min, and the various antibodies were configured according to a certain volume ratio with PBS as the diluent.

(5) 1 mL of FACS Buffer was added to terminate the reaction and centrifuged at a relative centrifugal force of 400 g for 5 min, and then the supernatant was discarded.

(6) 200 μl of fixative was added to each sample, overnight.

(7) The next day, 1 mL of perm buffer was added to the sample and centrifuged at a relative centrifugal force of 800 g for 10 min, and then the supernatant was discarded.

(8) 50 μl of a mixture of antibodies coupled with the metal isotopes against intracellular antigens was added to be stained and incubated on ice for 30 minutes.

(9) 1 mL of perm buffer was added and centrifuged at a relative centrifugal force of 800 g for 10 min, and then the supernatant was discarded.

(10) 1 mL of FACS Buffer was added and centrifuged at a relative centrifugal force of 800 g for 10 min, and then the supernatant was discarded, which was repeated for two times.

(11) 1 mL of deionized water was added and centrifuged at a relative centrifugal force of 800 g for 10 min, and then the supernatant was discarded.

(12) A cell counting plate was used for cell counting.

(13) 1 mL of deionized water was added and centrifuged at a relative centrifugal force of 800 g for 10 min, and then the supernatant was discarded.

(14) 20% EQ beads water was added for resuspending, ready for being loaded to the machine.

In the above steps, the buffer for flow cytometry assay (FACS Buffer) is the PBS containing 0.5 g of bovine serum albumin (BSA) and 0.02 g of sodium azide (NaN₃) per 100 mL.

In the above steps, the formula of the antibody blocking solution is per mL of PBS contains 20 mg of total mouse/hamster/rat IgG; the antibodies against intracellular antigens are anti-KI67, anti-iNOS, anti-Argnase I, anti-Foxp3, anti-CTLA-4.

In the above steps, the corresponding antibody is diluted in following multiples:

| Antibody | Dilution multiple |
|---|---|
| anti-CD45 | 1:200 |
| anti-CD44 | 1:200 |
| anti-CD19 | 1:100 |
| anti-KI67 | 1:200 |
| anti-CD24 | 1:100 |
| anti-MHC II | 1:400 |
| anti-B220 | 1:200 |
| anti-CD5 | 1:400 |
| anti-CD43 | 1:400 |
| anti-CD38 | 1:200 |
| anti-Ly6G | 1:200 |
| anti-Ly6C | 1:400 |
| anti-CX3CR1 | 1:100 |
| anti-IgD | 1:400 |
| anti-CD62L | 1:400 |
| anti-CD11c | 1:200 |
| anti-TCRγδ | 1:100 |
| anti-CD49a | 1:100 |
| anti-CD80 | 1:100 |
| anti-BST2 | 1:100 |
| anti-CD25 | 1:50 |
| anti-CD3 | 1:50 |
| anti-F4/80 | 1:100 |
| anti-CD115 | 1:100 |
| anti-iNOS | 1:100 |
| anti-CXCR3 | 1:100 |
| anti-CD27 | 1:100 |
| anti-CD103 | 1:100 |
| anti-ICOS | 1:100 |
| anti-Argnase I | 1:100 |
| anti-CD49b | 1:100 |
| anti-Foxp3 | 1:100 |
| anti-CD127 | 1:100 |
| anti-CD21 | 1:200 |
| anti-CD23 | 1:100 |
| anti-CD138 | 1:100 |
| anti-CD172a | 1:100 |
| anti-CTLA-4 | 1:100 |
| anti-SiglecF | 1:100 |
| anti-IgM | 1:100 |
| anti-CD4 | 1:800 |
| anti-CD8a | 1:400 |
| anti-CD11b | 1:100 |

IV. Data analysis

The data from mass spectrometry flow cytometry were analyzed using t-SNE and X-shift. The expressions of 43 detection antibodies in different cell subpopulations were distributed on a heat map, and the expressions of different markers and the distribution of different cell subpopulations were represented by viSNE plots.

Mass spectrometry flow cytometry analysis of the labeled whole immune cells in lung

As antibody labeled with metal isotope recognized and binded antigen on the surface or inside of cell, cells with the antibody labeled with the metal isotope were sent one by one to a plasma torch for ionization, causing a release of the tag of metal ion. The released metal ions were sent to a time-of-flight detection chamber for separation and detection, where the detector may record the precise time of arrival of the various ions, which in turn was converted into the exact amount of various metal labels in each cell, thereby obtaining the expression amount of antigens on the surface or inside of cell. The dimension reduction process was performed, and data from the mass spectrometry flow cytometry were then analyzed by t-SNE and X-shift. The expressions of the 43 detection antibodies were distributed on a heat map, and the expressions of different markers and the distribution of different cell subpopulations were represented by viSNE plot.

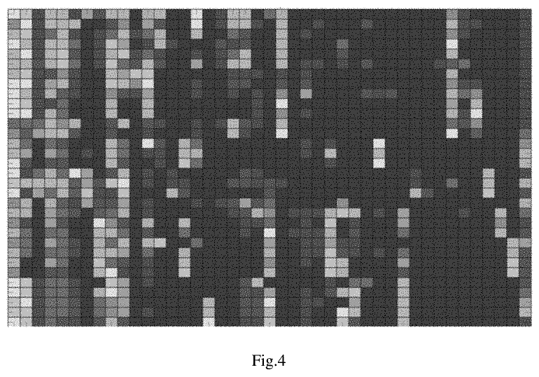

In FIG. 3, different shades of colors were arranged to represent the distribution of different cell subpopulations (the numbers in the figure refer to the cell subpopulations obtained by dimensionality reduction analysis and are not used as attached figure marks in the present invention, so they are not illustrated). In FIG. 4, the different color blocks represent the distribution of the expressions of 43 antibodies in different cell subpopulations.

Immune cell subpopulation

Based on the expression of markers on the cell surface, 32 cell subpopulations are obtained, covering CD45⁺CD3⁺ T cells, CD45⁺CD19⁺ B cells, CD45⁺CD49b⁺ NK cells, and CD45⁺CD11b⁺CD3⁻CD19⁻ myeloid cells. Mouse lung NK cells mainly comprise two cell subpopulations: lung NK cells expressing CD27 without CD49b, and lung NK cells expressing CD27 and CD49b; CD45⁺CD3⁻CD19⁻CD49b⁻ myeloid cells classified as (1) CD11b⁻MHCII⁻CD11c⁺F4/80⁺; (2) CD11b⁻CD103⁺; (3) CD11b⁺Ly6G⁺; (4) CD11b⁺Ly6C⁺CD11c⁻; (5) CD11b⁺Ly6C⁺CD11c⁺; (6) CD11b⁺Ly6C⁻CD11c⁺; (7) CD11b⁺Ly6C⁻CD11c⁻; granulocytes divided into eosinophils and two groups of neutrophils, wherein the neutrophils are divided into CD172a⁺ neutrophils and CD172a⁻ neutrophils based on CD172a expression. T cell population comprises CD4⁺ T cells, CD8⁺ T cells, CD25⁺ Treg, and γδ T cells. B cells are divided into IgM⁺ cell subpopulation and IgM⁺IgD⁺ cell subpopulation.

What is claimed is:

1. A method for establishing a characteristic atlas of whole immune cells in lungs of mice with acute lung injury, comprising:
(1) extraction of the whole immune cells in lung from the mouse:
taking a fresh mouse carcass that died naturally due to acute lung injury, removing the lung after irrigation and drainage of blood inside the lung; cutting up and digesting the lung with an enzyme mixture for half an hour, then performing density gradient centrifugation and erythrocyte lysis to obtain pure whole immune cells in lung of the mouse;
(2) labeling of antibody for mass cytometry:
binding the antibody against immune cell marker in lung of the mouse with stable metal isotope by using the MaxPAR X8 antibody coupling kit from Fluidigm, USA, to obtain a labeled antibody;
(3) staining the immune cells with the antibody:
incubating the isolated whole immune cells in lung of the mouse with the labeled antibody to label the immune cells;
(4) Mass cytometry analysis:
loading and analyzing the labeled whole immune cells in lung of the mouse on a mass cytometry, and analyzing the obtained data by t-SNE and X-shift algorithms; then distributing expressions of a plurality of detection antibodies in different cell subpopulations on a same heat map, and representing expressions of different markers and distribution of different cell subpopulations by viSNE plot, thereby showing a classification atlas of the whole immune cells in lung of the mouse.

2. The method according to claim 1, wherein the step (1) specifically comprises:
(1.1) wiping the mouse carcass with 75% ethanol cotton and cutting open the chest;
(1.2) continuously perfusing and flushing the lung with PBS through the heart to remove blood inside the lung and change the lung from blood red to white;
(1.3) isolating the lung from the mouse and placing in a culture dish containing PBS for washing by immersion;
(1.4) cutting up the lung and placing in a dissociation tube containing 2.4 mL of dulbecco's modified eagle medium (DMEM) and 0.3 mL of enzyme mixture;
(1.5) placing the dissociation tube in the GentleMACS™ Dissociator dissociation machine from MACS, Germany, with running twice in a m_lung_01 mode;
(1.6) removing the dissociation tube and placing on a shaker at a constant temperature of 37° C. and 220 rpm for 30 minutes for digestion;
(1.7) after digestion, placing the dissociation tube in the dissociation machine again with running once in a m_lung_02 mode to obtain a suspension;
(1.8) filtering the suspension in the dissociation tube through a 100 μm filter into a 15 mL centrifuge tube, and resuspending the dissociation tube with 2.5 mL of PBS to obtain a liquid portion, filtering the liquid portion into the centrifuge tube again;
(1.9) centrifuging at a relative centrifugal force of 300 g for 10 minutes at room temperature to obtain a first supernatant and a first precipitate;
(1.10) discarding the first supernatant to obtain the first precipitate, and adding 3 mL of 36% Percoll cell separation solution to the first precipitate; centrifuging at a relative centrifugal force of 450 g for 5 minutes at room temperature to obtain a second supernatant containing lung cell debris and a second precipitate;
(1.11) discarding the second supernatant containing lung cell debris to obtain the second precipitate, and adding 3 mL of ACK lysis buffer to the second precipitate for 3 minutes to completely remove erythrocytes; then adding 5 mL of PBS to terminate lysis of erythrocytes and centrifuging at a relative centrifugal force of 400 g for 5 minutes at 4° C. to obtain a third supernatant and whole immune cells; discarding the third supernatant to obtain the pure whole immune cells in lung of the mouse.

3. The method according to claim 2, wherein the PBS contains sodium chloride, potassium dihydrogen phosphate, disodium hydrogen phosphate and potassium chloride at a total concentration of 0.01 mol/L and a pH of 7.4.

4. The method according to claim 2, wherein the enzyme mixture is prepared by adding 12 mg of collagenase IV, 30 mg of pronase and 5 mg of deoxyribonuclease I powder to 100 mL of PBS and mixing well.

5. The method according to claim 2, wherein the dissociation tube containing the dulbecco's modified eagle medium and the enzyme mixture is preheated in a water bath at 37° C. for 5 minutes prior to placing the cut lung in the dissociation tube.

6. The method according to claim 2, wherein the Percoll cell separation solution is prepared by mixing 1 mL of 10× PBS with 9 mL of original Percoll solution well and then adding 15 mL of 1× PBS to obtain 25 mL of 36% Percoll separation solution.

7. The method according to claim 6, wherein the 10× PBS contains sodium chloride, potassium dihydrogen phosphate, disodium hydrogen phosphate and potassium chloride at a total concentration of 0.1 mol/L and a pH value of 7.4.

8. The method according to claim 2, wherein the centrifuge should be adjusted both ascending and descending speeds to the lowest setting before turning on the centrifuge in the steps (1.9) and (1.10).

9. The method according to claim 1, wherein the step (2) specifically comprises: labeling a multimer with a metal isotope to obtain a multimer chelated to a specific metal firstly, and then labeling an antibody with the multimer chelated to the specific metal to obtain the labeled antibody.

10. The method according to claim 1, wherein in the step (2), the stable metal isotope comprises:
Y-89, In-113, In-115, La-139, Pr-141, Nd-142, Nd-143, Nd-144, Nd-145, Nd-146, Nd-148, Nd-150, Sm-147, Sm-149, Sm-152, Sm-154, Eu-151, Eu-153, Gd-155, Gd-156, Gd-157, Gd-158, Gd-160, Gd-197, Tb-159, Dy-161, Dy-162, Dy-163, Dy-164, Ho-165, Er-166, Er-167, Er-168, Er-170, Tm-169, Yb-171, Yb-172, Yb-173, Yb-174, Yb-176, Lu-175, Pt-198, Bi-209;
the antibody comprises 43 antibodies, comprising: anti-CD45, anti-CD44, anti-CD19, anti-KI67, anti-CD24, anti-MHC II, anti-B220, anti-CDS, anti-CD43, anti-CD38, anti-Ly6G, anti-Ly6C, anti-CX3CR1, anti-IgD, anti-CD62L, anti-CD11c, anti-TCRγδ, anti-CD49a, anti-CD80, anti-BST2, anti-CD25, anti-CD3, anti-F4/80, anti-CD115, anti-iNOS, anti-CXCR3, anti-CD27, anti-CD103, anti-ICOS, anti-Argnase I, anti-CD49b, anti-Foxp3, anti-CD127, anti-CD21, anti-CD23, anti-CD138, anti-CD172a, anti-CTLA-4, anti-SiglecF, anti-IgM, anti-CD4, anti-CD8a, anti-CD11b.
